(19) United States
(12) Patent Application Publication
(10) Pub. No.: US 2021/0022638 A1
(43) Pub. Date: Jan. 28, 2021

(54) METHOD OF GENERATION OF A STATE INDICATOR OF A PERSON IN COMA

(71) Applicants: PARIS SCIENCES ET LETTRES - QUARTIER LATIN, Paris (FR); CENTRE NATIONAL DE LA RECHERCHE SCIENTIFIQUE, Paris (FR); ASSISTANCE PUBLIQUE HOPITAUX DE PARIS, Paris (FR); ÉCOLE NORMALE SUPÉRIEURE, Paris (FR); UNIVERSITÉ DE PARIS, Paris (FR); INSERM (INSTITUTE NATIONAL DE LA SANTÉ ET DE LA RECHERCHE MÉDICALE), Paris Cedex 13 (FR)

(72) Inventors: David HOLCMAN, Paris (FR); Adrien DOUMERGUE, Saint-Maur-Des-Fossés (FR); Nathalie KUBIS, Paris (FR); Alexandra RICHARD, Paris (FR); Aymeric FLOYRAC, Arcueil (FR)

(73) Assignees: PARIS SCIENCES ET LETTRES - QUARTIER LATIN, Paris (FR); CENTRE NATIONAL DE LA RECHERCHE SCIENTIFIQUE, Paris (FR); ASSISTANCE PUBLIQUE HOPITAUX DE PARIS, Paris (FR); ÉCOLE NORMALE SUPÉRIEURE, Paris (FR); UNIVERSITÉ DE PARIS, Paris (FR); INSERM (INSTITUT NATIONAL DE LA SANTÉ ET DE LA RECHERCHE MÉDICALE), Paris Cedex 13 (FR)

(21) Appl. No.: 16/982,174

(22) PCT Filed: Mar. 22, 2019
(86) PCT No.: PCT/FR2019/050664

§ 371 Date: Sep. 18, 2020

(30) Foreign Application Priority Data
Mar. 22, 2018 (FR) 1852473

(51) Int. Cl. A61B 5/0484; A61B 5/04; A61B 5/00

(52) U.S. Cl. CPC A61B 5/04845; A61B 5/7275; A61B 5/7264; A61B 5/04014

(57) ABSTRACT

This method for generating an indicator of the state of a patient in coma includes: generating at least one auditory stimulation by generating a sequence of auditory stimuli, the sequence producing evoked potentials in the patient; acquiring a first electroencephalographic signal produced by patient from at least one electrode; estimating at least one pair of values corresponding to a first parameter and a second parameter extracted from the first acquired signal, including estimating a first pair of values such that calculating the first parameter includes an estimation of the amplitude variance of the first signal within a predefined time window and the calculation of the second parameter includes an estimation of the correlation of two segments of the first signal; generating a state indicator for the or each pair of values of the first and second parameters, the values defining coordinates of a point in a reference base.

### FIELD OF THE INVENTION

The present invention relates to methods of generating an indicator for assessing the likelihood of awakening of a patient in a coma. The invention is particularly applicable to the analysis of electrophysiological signals and more particularly of electroencephalographic signals. The field of the invention relates to methods of generating graphical indicators and to the representation of these indicators in a two-dimensional graph.

### STATE OF THE ART

There is an interest in evaluating the condition of patients in a coma, and in particular in prognosing the probabilities of awakening of the clinical case treated. Post-anoxic encephalopathy after cardiac arrest is a frequent cause of admission for coma in intensive care. From this study base of patients in post-anoxic coma, treatment and trial modalities were carried out. One challenge is to predict the possibilities of awakening of a patient and to assess neurological damage in order to establish a neurological prognosis. Among the evaluation methods, there is a clinical evaluation performed by the physician and possibly an electrophysiological evaluation based on analyzes of physiological signals, for example, in response to stimuli. There are also evaluations carried out using biological markers, for example markers of the S100 beta or NSE proteins type.

Post-anoxic encephalopathy and its prognosis is assessed by electroencephalogram, called EEG. The absence of a N20 response to somesthetic evoked potentials after median nerve stimulation has almost 100% specificity for predicting no awakening in adults. However, the real lack of response in patients in intensive care is difficult to assert due to the electrical environment which generates numerous artefacts thus making it extremely difficult to interpret the low amplitude response of the evoked potential in the child and a patient suffering from head trauma.

One possible solution to improve the prognosis for awakening of patients is based on the analysis of auditory evoked potentials. Auditory evoked potentials are recorded with electrodes placed on the scalp reflecting the cerebral response to repeated and averaged auditory stimulions. The “MisMatch Negativity” method, known as MMN, makes it possible to represent a cerebral integration of automatic detection of these infrequent and random stimulations in a continuous series of sounds.

However, the analysis and recognition of the presence or absence of an indicator that can predict a patient's awakening is difficult in intensive care patients. Indeed, in this case, numerous artefacts as well as a possible drastic reduction in the amplitude of the evoked potentials make the visual processing of the signals difficult. Another drawback of this method is the loss of information inherent in the averaging mechanisms of the acquired signals.

In any case, the MMN method does not represent a reliable or easy to use predictive method in intensive care units either. It is therefore desirable to define a method making it possible to generate a predictive indicator of the state of a patient in a coma, in particular to assess a probability of arousal.

### SUMMARY

The present invention relates to a method of generating a state indicator of a given patient in a coma, comprising the following steps:
generation of at least one auditory stimulation by generating a sequence of auditory stimuli, said sequence producing evoked potentials in the given patient;
acquisition of a first electroencephalographic signal produced by said given patient from at least one electrode;
estimation of at least one pair of values corresponding to a first parameter and a second parameter extracted from the first acquired signal, the calculation of the first parameter comprising an estimate of the amplitude variance of the first signal in a predefined time window, the calculation of the second parameter comprising an estimate of the correlation of two segments of the first signal;
generation of a state indicator defined by the pair of values of the first and second parameters, said values defining coordinates of a point in a reference base.

One advantage is to determine a representation in which an indicator of a patient's state can allow him to be classified against a repository comprising other state indicators corresponding to other patients.

According to one embodiment, at least one stimulation comprises at least one sequence of auditory stimuli comprising at least one periodic pattern of predefined frequency.

One advantage is to determine a sequence to optimize the relevance of the indicator. In addition, such a stimulus is easily reproducible and can serve as a reference for a set of patients.

According to one embodiment, the calculation of the first parameter comprises the following steps:
filtering of the first acquired signal;
filtrage du premier signal acquis;
segmentation of the first signal according to a first time window in order to generate a plurality of epochs synchronized with the predefined frequency, said epochs being averaged over the same time window;
extraction of an epoch averaged in a second predefined time window, each of said epochs having a predefined duration and being synchronized with a stimulus of at least one sequence of auditory stimuli;
calculation of the amplitude variance of the signal averaged over the second predefined window.

One advantage of filtering is to obtain a signal with improved processing. The advantage of segmentation is to determine a portion of the signal which is possible to average. An advantage of averaging is to remove a set of artifacts that can interfere with the variance calculation.

According to one embodiment, the calculation of the second parameter comprises the following steps:
selection of at least a first and a second signal segment from the acquired electroencephalographic signal, said first and second segments having the same duration;
first segmentation of the first signal segment over a third time window generating a plurality of epochs of duration corresponding to the duration of the third window, each of said epochs having a predefined duration and being synchronized with a stimulus of at least one sequence of auditory stimuli;
second segmentation of the second signal segment over the third time window generating a plurality of epochs of duration corresponding to the duration of the third window, each of said epochs having a predefined duration and being synchronized with a stimulus of at least one sequence of auditory stimuli;
generation of a first signal resulting from the averaging of the epochs of the first segmentation and generation of a second signal resulting from the averaging of the epochs of the second segmentation;
generation of the second parameter from the calculation of the time correlation between the first average signal and the second average signal over a fourth time window.

According to one embodiment, the first time window and the third time window have the same duration, corresponding to the inverse of the predefined frequency.

One advantage is to homogenize the representation of the pair of selected points.

According to one embodiment, the second time window and the fourth time window have substantially equal durations ranging from 20 ms to 320 ms. According to one example, the duration of the first and second segments is ranging from 5 min to 25 min, preferably of the order of 10 min.

One advantage is to define a time window specially adapted to the responses to auditory stimuli.

According to one embodiment, the generation of the state indicator for said given patient comprises the calculation of a probability that said state indicator belongs to a predefined class of states from a Gaussian estimator and the rules from Bayes.

According to one embodiment, the generation of the state indicator for said given patient comprises calculating a probability that said state indicator belongs to a predefined class of states from the k-neighbor method.

According to one embodiment, the method comprises a step of measuring a first distance between said state indicator and a first set of points having coordinates represented in the same reference base and of measuring a second distance between said state indicator and a second set of points having coordinates represented in the same reference base.

According to one embodiment, the method comprises a step of comparing the first distance and the second distance.

According to one embodiment, the state indicator is associated with a probability calculated from a probability classification model or a supervised learning classification method.

According to one embodiment, the classification comprises two classes.

According to one embodiment, the steps are repeated for a second patient and/or any other patient associated with an electroencephalographic signal or with data recorded from a database and further include:
a) generating a graph for a plurality of coordinates from a patients population, in which each patient is associated with coordinates;
b) identifying at least one region of interest of the graph for which a subset of selected patients shares the same class of the predefined classification;
c) associating of a probability to one of the classification classes for each of the identified regions of interest; and
d) generating a probability associated with the state indicator of the first patient based on the coordinates.

According to one embodiment, for the constitution of a baseline for the evaluation of a probability of awakening of a patient in a coma, said method comprises:
a) generating a state indicator for a new patient;
b) recording the set of coordinates in a memory, said coordinates being associated with a state indicator of said first patient;
c) updating a set of wake-up reference data stored in a memory.

The present invention further relates to a device comprising a memory for storing data, in particular coordinates of a graph that have been calculated beforehand, and a calculator for performing operations on signals acquired by a measuring means such as an electrode, said calculator making it possible in particular to perform operations of comparison, of averaging or of correlation of signals, characterized in that it implements the steps of the method.

The present invention further relates to a device for generating a state indicator of a given patient in a coma, comprising:
a stimulation module configured to generate at least one auditory stimulation by generating a sequence of auditory stimuli, said sequence producing evoked potentials in the given patient;
an acquisition module configured for the acquisition of a first electroencephalographic signal produced by said given patient from at least one electrode;
a calculation module configured for estimating at least one pair of values corresponding to a first parameter and a second parameter extracted from the first acquired signal, comprising the estimation of a first pair of values such that the calculation of the first parameter comprises an estimate of the amplitude variance of the first signal in a predefined time window and the calculation of the second parameter comprises an estimate of the correlation of two segments of the first signal;
a generation module configured to generate a state indicator for the or each pair of values of the first and second parameters, said values defining coordinates of a point in a reference base.

According to one embodiment, at least one stimulation generated by the stimulation module comprises at least one sequence of auditory stimuli comprising at least one periodic pattern of predefined frequency.

According to one embodiment, the calculation module is configured to calculate the first parameter of the first pair of values according to the following steps:
filtering the first acquired signal;
segmentation of the first signal according to a first time window in order to generate a plurality of epochs synchronized with the predefined frequency (1 s), said epochs being averaged over the same time window;
extracting an epoch averaged in a second predefined time window [20 ms-320 ms], each of said epochs having a predefined duration and being synchronized with a stimulus of at least one sequence of auditory stimuli;
calculating the amplitude variance of the signal averaged over the second predefined window.

According to one embodiment, the calculation module is configured to calculate the second parameter of the first pair of values according to the following steps:
selection of at least a first and a second signal segments from the acquired electroencephalographic signal, said first and second segments having the same duration;
first segmentation of the first signal segment over a third time window (1 s) generating a plurality of epochs of duration corresponding to the duration of the third window, each of said epochs having a predefined duration and being synchronized with a stimulus of at least one sequence of auditory stimuli;
second segmentation of the second signal segment over the third time window (1 s) generating a plurality of epochs of duration corresponding to the duration of the third window, each of said epochs having a predefined duration and being synchronized with a stimulus of at least one sequence of auditory stimuli;
generation of a first signal X(t) resulting from the averaging of the epochs of the first segmentation and generation of a second signal Y(t) resulting from the averaging of the epochs of the second segmentation;
generation of the second parameter from the calculation of the time correlation R(X, Y) between the first average signal X(t) and the second average signal Y(t) over a fourth time window ([20 ms-320 ms]).

According to one embodiment, the calculation module is configured for estimating a second pair of values extracted from the first acquired signal, such that the calculation of the first parameter comprises an estimate of the number of local extremums in the first signal in a predefined time window and the calculation of the second parameter comprises the sum of the absolute values of the potential value differences of the first signal between two successive local extremes in a predefined time window, allowing the generation of a second state indicator defined by the second pair of values of the first and second parameters.

According to one embodiment, the first time window and the third time window have the same duration, corresponding to the inverse of the predefined frequency.

According to one embodiment, the generation module is configured to generate the state indicator for said given patient from the calculation of a probability that said state indicator belongs to a predefined class of states from a Gaussian estimator, Bayes rules and/or a support vector machine.

According to one embodiment, the generation module is configured to generate the state indicator for said given patient from the calculation of a probability that said state indicator belongs to a predefined class of states from the k nearest neighbors method.

According to one embodiment, the generation module is configured to generate the state indicator for said given patient from the calculation of a probability that said state indicator belongs to a predefined class of states from the minimum between the probabilities estimated from the k nearest neighbors method, the weighted k nearest neighbors method, the Gaussian estimator, Bayes' rules and/or the support vector machine.

According to one embodiment, the generation module is configured to generate the state indicator for said given patient from the calculation of a probability that the first state indicator belongs to a predefined class of states from the k nearest neighbors method and the calculation of a probability that the second state indicator belongs to a predefined class of states from the weighted k nearest neighbors method.

According to one embodiment, the probability that the patient belongs to a predefined state class is estimated as the minimum between the probability calculated for the first indicator from the k nearest neighbors method and the probability calculated for the second indicator state from the weighted k nearest neighbors method.

According to one embodiment, a calculation module is configured to measure a first distance between said state indicator and a first set of points having coordinates represented in the same reference base and for measuring a second distance between said indicator. state and a second set of points having coordinates represented in the same reference base.

According to one embodiment, a calculation module is configured to compare the first distance and the second distance.

According to one embodiment, the state indicator is associated with a probability calculated from a probability classification model or a supervised learning classification method.

According to one embodiment, the classification comprises two classes.

According to one embodiment, a calculation module is configured to repeat the steps for a second patient and/or any other patient associated with an electroencephalographic signal or with data recorded from a database and to further implement the following steps:
generating a graph for a plurality of coordinates from a patients population, in which each patient is associated with coordinates [(P1); (P2)]ᵢ;
identifying at least one region of interest of the graph for which a subset of selected patients shares the same class of the predefined classification;
associating a probability with one of the classification classes for each of the identified regions of interest;
generating a probability associated with the state indicator of the first patient based on the coordinates [(P1); (P2)].

The present invention further relates to a system comprising a generator of auditory stimuli emitted with a predefined period for a predefined duration and a set of electrodes for measuring a cerebral electrical activity of a patient, this system comprising a device for the generation of a state indicator of said patient as described above.

### Definitions

The term “subject” refers to a mammal, preferably a human. In one embodiment, a subject may be a “patient”, i.e. a warm blooded animal, more preferably a human, who is awaiting reception or is receiving medical care, or has been/is/will be undergoing a medical procedure, or is being watched for the development of a disease. In one embodiment, the subject is an adult (e.g. a subject over the age of 18). In another embodiment, the subject is a child (e.g. a subject under 18). In one embodiment, the subject is a man. In another embodiment, the subject is a woman.

The term “stimulus”, or “stimuli”, designates any physical, chemical or biological element or any other event, such as an audible or sound or visual event, capable of triggering phenomena in the organism, in particular electrical phenomena, electrophysiological, nervous, muscular or endocrinal. More particularly, in the context of the invention, a stimulus or stimuli will be sound sequences broadcast to a patient in a coma.

The term “coma” refers to a physiological state of a subject, or patient, who has lost consciousness. It more particularly designates a prolonged loss of consciousness and/or alertness.

### BRIEF DESCRIPTION OF THE FIGURES

FIG. 1 is a diagram of an embodiment of the system of the invention, showing the main elements implementing the steps of the method of the invention.

FIG. 2 is a two-dimensional graph according to an embodiment of the invention including a distribution of the probabilities of awakening according to the invention in which a point generated by the method of the invention is displayed.

FIG. 3 is a flowchart of an embodiment of the invention in which the main steps of the method of the invention are represented, this embodiment involving a step of comparing data from a patient with a corpus of recorded data.

FIG. 4 is a flowchart of an embodiment of the invention in which the main steps of the method of the invention are represented, this embodiment involving a step of graphical representation of data from a patient with a corpus of data generated on a same representation.

FIG. 5A is a representation of a signal acquired within the framework of the method of the invention.

FIG. 5B is a representation of a filtered signal resulting from filtering the signal of FIG. 5A according to a step of the method of the invention.

FIG. 5C is a representation of an averaged signal resulting from the averaging of the filtered signal of FIG. 5B according to a step of the method of the invention.

FIG. 5D is a representation of a signal resulting from the average of signals acquired from multiple electrodes.

FIG. 5E is a representation of a filtered signal resulting from filtering the average signal of FIG. 5D according to a step of the method of the invention.

FIG. 5F is a representation of an average signal resulting from the average in a 500 ms window of the filtered signal of FIG. 5E according to a step of the method of the invention.

FIG. 6A is a two-dimensional graph according to an embodiment of the invention comprising a distribution of the probabilities of awakening according to a Gaussian-Bayesian classification method in which a point (s(X), R(X, Y)) generated by the method of the invention is displayed.

FIG. 6B is a two-dimensional graph according to an embodiment of the invention comprising a distribution of the probabilities of awakening according to a k-cclassification method in which a point (s(X), R(X, Y)) generated by the method of the invention is displayed.

FIG. 6C is a two-dimensional graph according to an embodiment of the invention comprising a distribution of the probabilities of awakening according to a Gaussian-Bayesian classification method in which a point (N_{E}, |ΔV|) generated by the method of the invention is displayed.

FIG. 6D is a two-dimensional graph according to an embodiment of the invention comprising a distribution of the probabilities of awakening according to a k-Neighbors classification method in which a point (N_{E}, |ΔV|) generated by the method of invention is displayed.

### DETAILED DESCRIPTION

The following description will be better understood when read in conjunction with the drawings, in which preferred embodiments of the invention are shown, for illustrative purposes only. Of course, the present application is not limited to the arrangements, structures, characteristics, embodiments or appearances described and shown. The drawings are not drawn to scale and are not intended to limit the scope of the claims to the embodiments depicted in these drawings only. Accordingly, it should be understood that where features mentioned in the appended claims are followed by reference signs, such signs are included solely for the purpose of enhancing the intelligibility of the claims and are in no way limiting on the scope of the claims.

In the present description, we will speak interchangeably of awakening or waking up from a coma of a given patient. We will deal with the general case of a “state indicator”, which can be interpreted in a case of application of the invention to an “awakening indicator”. Further, the exemplary embodiment describes two state classes C₁, C₂, but according to other exemplary embodiments, several state classes may be compatible with the invention.

FIG. 1 shows an embodiment of the system of the invention.

The system of the invention comprises an auditory stimulation generator GEN_SA. The generated GSA auditory stimuli are preferably repeated or transmitted periodically. For this purpose, a loudspeaker, a speaker or any other device making it possible to emit a sound or a sound sequence can be used. The GEN_SA generator is preferably placed next to a patient H during its use.

The system of the invention comprises a set of ELEC electrodes intended to be affixed in contact with a patient, such as a patient H. The ELEC electrodes are intended to measure a brain activity of patient H. They make it possible in particular to acquire signals S₁, Sᵢ, Sₙ and deliver the acquired signals to a calculator K.

The system of the invention comprises said calculator K. The latter is configured to execute instructions in order to deduce output parameters of the acquired signals S₁, Sᵢ, Sₙ by the ELEC electrodes.

The system of the invention further comprises an AFF display for displaying points in a representation of probability distributions of awakening of a plurality of patients who have been in a coma or are still in a coma. Thus, the AFF display makes it possible to display previously acquired processed data from a plurality of patients H who have been in a coma. These data make it possible to consolidate a distribution of probabilities of awakening or not of a set of patients.

Consequently, the AFF display makes it possible to directly deduce a conclusion by reading the position of the point generated within the probability distribution. The comparison of a point generated with the position of the other points can be carried out with the naked eye by an operator or can be carried out automatically by means of a calculator from the calculation of the distance between said points.

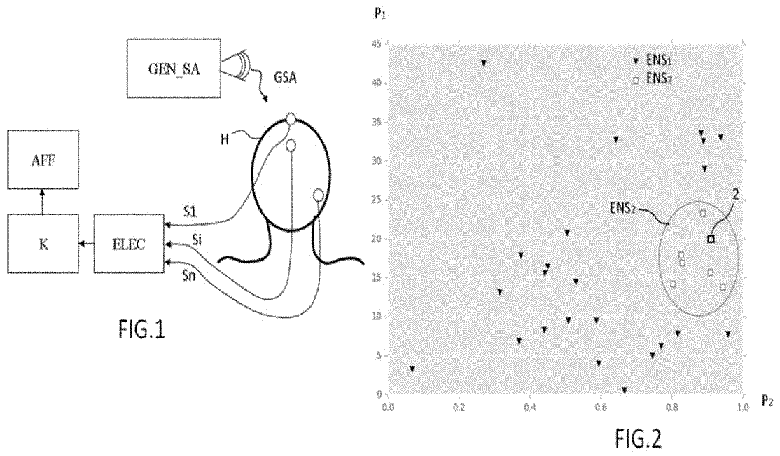

One of the advantages of the invention is to generate a graph in a frame whose axes correspond to parameters deduced from the signals acquired. FIG. 2 gives an example of a possible representation of an awakening probability distribution. The COORD coordinate system used can be defined on the abscissa and ordinate by spectral parameters or resulting from a processing of the signals acquired by the electrodes. The parameters can result from one or more operations on the acquired signals, namely averages, maximum values, standard deviations, variances, correlations, or even comparisons or superimpositions of acquired signals.

Furthermore, the invention relates to a method for estimating an indicator of awakening of a patient in a coma.

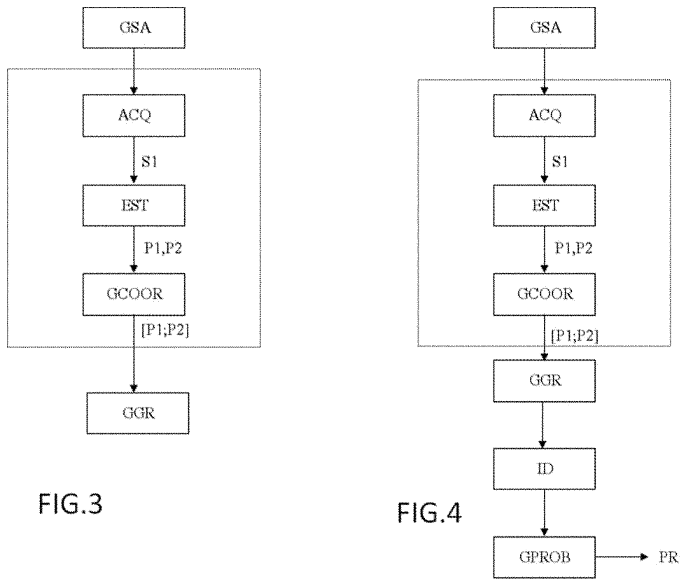

According to one embodiment, the method comprises generating at least one auditory stimulation by generating a sequence of GSA auditory stimuli. The sequence produces evoked potentials in the given patient H in a coma. FIG. 3 represents this step denoted GSA.

The auditory stimulation is configured to induce a cognitive process in the stimulated subject. The human brain is able to extract patterns or regularities in its environment, for example object A is always followed by object B but never by object C. The brain can detect the probabilities of transition automatically, that is, even when the subject's attention is distracted or when the stimuli are presented below the threshold of consciousness. Automatic brain responses to a violation of a rule (or pattern) can also be detected if the stimuli are in a close or local temporal neighborhood (i.e. a few seconds). Incompatible responses can be produced with complex sequences such as a melody or a rhythm, even in unconscious subjects.

According to a first example, the auditory stimulation is caused by the emission of a predefined sequence of sounds. The sequence comprises, for example, a series of sounds emitted at regular intervals with the same spectral content. Sounds are emitted with a predefined frequency f₀. The repetition period can be for example 1 second. According to different embodiments, the period can range from a few milliseconds to several minutes.

According to an exemplary embodiment, the sound sequence comprises spectral patterns which evolve as a series of sounds ranging from high notes to low notes or vice versa. The sequence is then repeated over a period of sequences which may range from a few seconds to several hours. The transmitted sequence may contain noises of different frequencies. According to one embodiment, the sequence can be repeated identically with a few random and infrequent modifications (i.e. deviant stimuli), for example from 10 to 20% of the standard sequences.

According to one embodiment, the auditory stimulation comprises several auditory tests having a predefined interval between tests, each auditory test being formed of N consecutive auditory stimuli having a predefined duration with a predefined interval between the auditory stimuli. In this embodiment, the auditory stimulation has a first percentage of standard local tests comprising N identical auditory stimuli and a second percentage of locally deviant tests comprising the first N−1 identical auditory stimuli and the N^{th} auditory stimulus different from the previous N−1 auditory stimuli, where N is equal to or superior than 2. Such a sequence is therefore composed of a standard sound repeated a number of times, followed by a deviant sound. Comparison to a condition in which all sounds in the sequence are standard usually reveals the occurrence of the negativity of the mismatch. Crucially, if the deviant sequence is very common in a stimulation, subjects will expect the last sound to be deviant.

According to an exemplary embodiment, the sequence can comprise the emission of a succession of five identical sounds regularly spaced and of a note (ie non-deviant stimuli) or of a sound having a different spectral content (ie deviant stimulus). Thus, many sequences can be used within the scope of the invention.

One advantage of producing a wide variety of sequences is to test different sources of stimulation in order to constitute a library of sequences making it possible to be adapted to the use of the invention. Another advantage is that it allows comparable sequences to be produced from one patient to another in order to validate or invalidate particular sequences.

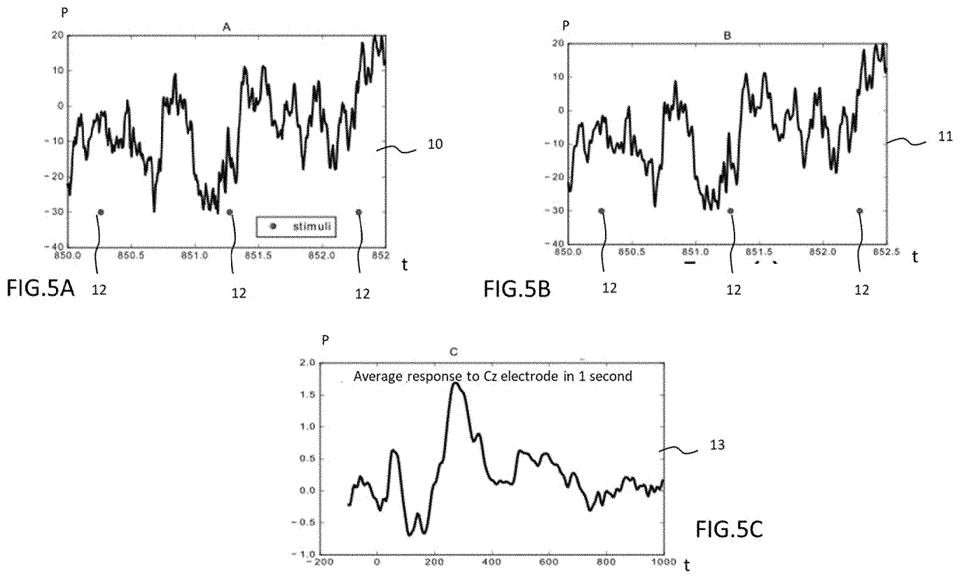

According to one embodiment, the method of the invention comprises the acquisition of an electroencephalographic signal, called EEG signal, produced by said patient from at least one system of electrodes comprising for example at least one active electrode and a reference electrode. FIG. 3 and FIG. 4 represent this step denoted ACQ.

According to one embodiment, the means for measuring the electroencephalographic signals comprise at least two surface electrodes. According to an exemplary embodiment, a first front reference electrode and a second central active electrode (Cz) are arranged on the cranial surface of a patient.

According to one embodiment, the means for measuring the electroencephalographic signals comprise at least the electrodes Fz, Cz, C3, C4, T3, T4 according to the 10-20 system.

According to one embodiment, the electrodes are electrodes intended to be mounted, for example, on a helmet which itself is intended to be worn by a patient.

According to one embodiment, the method comprises a step of estimating at least one pair of values corresponding to a first parameter P₁ and a second parameter P₂. This step is denoted EST in FIG. 3 and FIG. 4. The first parameter P₁ and the second parameter P₂ are extracted from the first acquired signal S₁ or from the signal processed after its acquisition, for example, by filtering or averaging. They can, for example, restore the properties of the signals over longer or shorter times. By way of example, the first parameter P₁ can be developed so as to restore a property of the signal over a short period corresponding for example to the duration of the cognitive tasks. The first parameter P₁ can then be perceived as a local property of the subject's responses. The second parameter P₂ can be developed or chosen by considering properties of the signal over longer periods.

The first parameter P₁ is estimated by performing various operations on an acquired signal S It can in particular be filtered, segmented, averaged in order to define a pattern in which a selection of an extract of the signal is used to calculate a variance in amplitude.

According to one embodiment, the method comprises a step of filtering the acquired EEG signals.

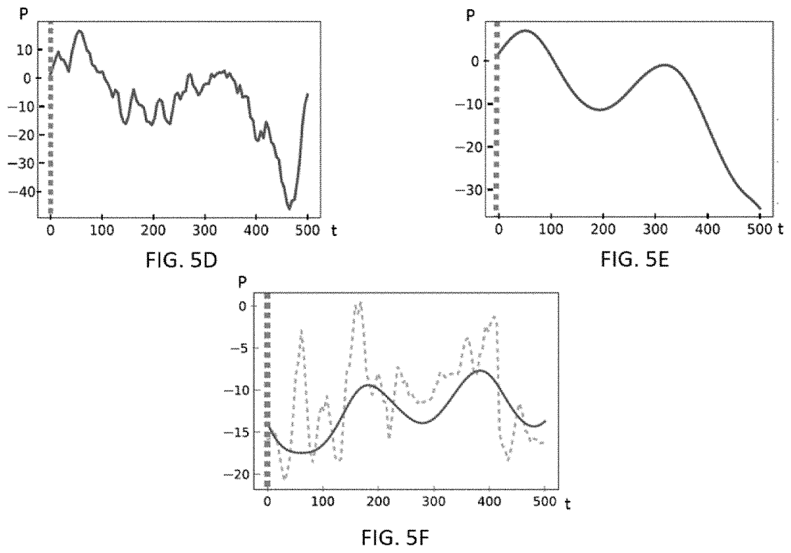

FIG. 5A illustrates a representation of an acquired EEG signal 10 in which appear, on the ordinate “P”, the values of the potentials at the Cz electrode, and on the abscissa “t”, the time in seconds. The graph also shows the moments at which the stimuli 12 are generated.

FIG. 5B represents the filtered signal 11. In this example, the signal is filtered with a Butterworth bandpass filter of rank n=4 on the frequency range 0.1-50 Hz.

FIG. 5C represents an averaged response 13 reduced to the time interval [0; 1 s] in order to define epochs. The time interval [0; 1 s] defines a first time window F1. This first window F1 can be configured according to the embodiment envisaged. In this example, the time t=0 corresponds to the moment when the auditory stimuli are produced. This preliminary step makes it possible to average the responses in order to bring out the response of the EEG to the stimuli. The extracted epochs are all synchronized with the frequency f₀ at which the stimulations are produced.

According to one embodiment, the filtering and averaging steps are steps involved for the estimation of the parameter P₁.

According to a first exemplary embodiment, an extracted parameter corresponds to the variance s(X) of the amplitude of the signal X(t) in the time interval of 20 to 320 ms. This interval defines a second time window F₂. The amplitude variance can be calculated from a calculation means such as a calculator.

The chosen timescale advantageously corresponds to response time scales of neural networks involved in cognitive tasks. According to other embodiments, the considered timescales can be more selective, for example from 60 ms to 200 ms.

According to another embodiment, the time interval is divided into different sub-ranges, for example [20; 120 ms], [120 ms; 220 ms], [220 ms; 320 ms]. This subdivision makes it possible, for example, to refine the analyzes by segmenting the responses of neural networks to differentiated cognitive tasks. According to one example, intermediate calculations of amplitude variances can be performed and then combined. According to a preferred embodiment, the calculation of the amplitude variance is carried out over the range of times from 20 ms to 320 ms.

According to one embodiment, the calculation of the amplitude variance accounts for the amplitude of the fluctuations represented by the basal activity of the neural networks. Variance is used to analyze the evoked signals generated by a response to standard periodic auditory stimuli.

FIG. 2 shows on the ordinate the parameter corresponding to the amplitude variance of the signal acquired or processed after filtering and averaging.

The averaging step makes the calculation more reliable because of the different measurements taken into account. The filtering step eliminates noise and artefacts that would not result directly from the electrical activity produced by the stimulations.

According to one embodiment, the step of estimating the parameters comprises estimating a parameter restoring a property of the signals over a longer period. This is an indicator resulting from a correlation of two segments SEG₁ and SEG₂ of the signals acquired or processed.

According to an exemplary embodiment, the acquisition time D_{A} is 20 minutes. In other examples, it can range from a few minutes to several minutes. Preferably, the chosen duration ranges from 10 min to 40 min.

According to one embodiment, the acquisition time D_{A} is segmented into two ranges of duration D1 substantially equal. For example the two ranges [1; 10] min and [10; 20] min can define two successive ranges making it possible to segment the first S₁ signal. According to other embodiments, the number of segmentations can be from three ranges to a few tens of ranges.

The method comprises a step of segmenting each segment of duration D₁ into a plurality of epochs segmented in a predefined time window. This is a third time window F₃. This window is advantageously of the same duration as the first window which made it possible to calculate the parameter P₁.

According to an exemplary embodiment, F₁=F₃=1 s, that is to say, in this example, the inverse of f₀.

The step of estimating the parameter P₂ comprises the averaging of the epochs over each of the segmented time windows defined in the previous paragraph.

According to one embodiment, the estimation of the second parameter P₂ comprises a generation of two signals, denoted X(t) and Y(t). According to an exemplary embodiment, the epochs are defined in the time interval [0; 1] s. According to other examples, the normalization of the window F₃ can be shorter or longer, such as for example half a second or two seconds.

The step of estimating the parameter P₂ comprises a calculation of the time correlation of these two signals by the function R(X, Y) over a time window F₄. According to one embodiment, the time window F₄ has a duration substantially equal to that of the second time window F₂, that is to say included in the interval [20, 320] ms. Advantageously, part of the signal averaged over the window F₃ is used to calculate the temporal correlation R(X, Y). The part used, as before, advantageously corresponds to the time scale of the neuronal response to stimulations.

According to an exemplary embodiment, the correlation function R can be written:

R(X,Y)=[<(X(t)−m(X))·(Y(t)−m(Y)>]/[<X(t)²><Y(t)²>],

where <.> represents the time average and m(X) the average of the variable X.

As explained previously, deviant stimuli represent approximately one tenth of evoked responses. According to one embodiment, to analyze these responses, the signals acquired from several electrodes are summed and then the obtained average signal is filtered and averaged over a predefined time window F5.

FIG. 5D illustrates a representation of an average signal resulting from the average of the signals acquired from electrodes Cz, C3, C4 and Fz as a function of time in seconds. The graph also shows the moment at which the deviant stimulus is generated, by a vertical line.

FIG. 5E represents an example in which the signal is filtered with a Butterworth low-pass filter of rank n=2 with a threshold at 10 Hz.

FIG. 5F shows an averaged response scaled to the time interval [0-500 ms] following the emission of a deviant stimulus.

According to one embodiment, at least two parameters are extracted from this average signal over the time interval [0; 500 ms]. According to a first exemplary embodiment, an extracted parameter corresponds to the number of local extremums N_{E} in said mean signal. According to a second exemplary embodiment, an extracted parameter corresponds to the sum of the absolute values of the differences, between two local and successive extremes eᵢ, of the potential of the average signal V(eᵢ) according to the formula |ΔV|=Σᵢ|V(eᵢ)−V(eᵢ₊₁)|.

The N_{E} and |ΔV| parameters, being calculated over a time window of 500 ms following a deviant stimulus, allow the subject's response to the deviant stimuli to be assessed.

According to one embodiment, the method of the invention makes it possible to define coordinates from a pair of values {P₁; P₂}. According to one example, the coordinate point {P₁; P₂} is generated in a reference base B_{REF}. According to one embodiment, the reference base constitutes a standardized reference frame. According to an exemplary embodiment, the reference base is materialized by a graph comprising a reference frame with two axes (Ox), (Oy) defining an abscissa and an ordinate.

According to one embodiment, the method of the invention makes it possible to define a first coordinate system from the pair of values {s(X), R(X, Y)} and/or a second coordinate system from the. pair of values {N_{E}, |ΔV|}. The first system is associated with responses to non-deviant stimuli and the second system is associated with responses evoked by deviant stimuli.

In the example of FIG. 2, the parameter P₂ is shown in abscissa and the parameter P₁ in ordinate. The coordinates of point 2 correspond to those of a new patient and are noted G_{COOR}.

According to an exemplary embodiment, the method of the invention comprises the generation of a graph comprising a plurality of points defining a reference base displayed on the same graph. FIG. 2 represents a first set of points ENS₁ defining black triangles and belonging to a first given class of states C₁ of patients, and a second set ENS₂ of points defining white squares and belonging to a second class C₂ of patients.

According to an exemplary embodiment, the two-dimensional representation of data is obtained by generating a point for a given patient H from the values of the parameters P₁ and P₂ which were obtained following a phase of auditive tests and measurement of brain activity. This step is denoted GGR in FIGS. 3 and 4.

According to a first exemplary embodiment, along the Oy axis, the amplitude variance s(X) is calculated over the entire 20-minute sample. According to the Ox axis, the correlation function R(X, Y) is calculated as detailed previously. Finally, a given point Pt has for coordinates: Pt=(x,y)=(s(X), R(X, Y)). According to a second exemplary embodiment, a given point Pt has for coordinates: Pt=(x,y)=(N_{E}, |ΔV|).

The invention therefore makes it possible to generate a map for a set of patients H. FIG. 2 thus represents a plurality of points, each point corresponding to a test carried out for a given patient H.

FIG. 2 represents a distribution of patients H having been in a coma or still being in a coma. In this example, two time windows of 10 min were configured with a measurement, in particular considering the Cz electrode. We find along the Ox axis, the correlation R(X, Y), and along the Oy axis, the standard deviation s(X) of the noise, that is to say fluctuations in the averaged EEG signal. Deceased patients are represented by black triangles and constitute the ENS₁ set. Survivors and awakened are represented by white squares and constitute the ENS₂ set.

The invention therefore makes it possible to visually detect to which set a new generated point 2 may belong. FIG. 3 shows the main steps of an embodiment of the method for generating a state indicator.

FIG. 4 represents an embodiment of the method of the invention comprising additional steps aimed at associating a probability with the state indicator.

According to one embodiment, the invention enables to assign the state indicator to a probability of awakening of patient H, that is to say a probability of coming out of a coma.

The method of the invention therefore includes a representation of probabilities on a graph making it possible to assign a given probability as a function of the position of point 2 in the graph.

When the graph includes areas identified as iso-probability, the method comprises the identification of the region of interest RI for which a subset of selected patients share an identical probability of belonging to the same class of the predefined classification, for example in the present case C₁ or C₂. This step is denoted ID in FIG. 4.

The method is therefore able to deduce an association between the point generated on the graph and a probability of awakening depending on the position of the point. The method can then include an automatic step for generating, denoted GPROB, a probability PR that the first patient H belongs to one class or another.

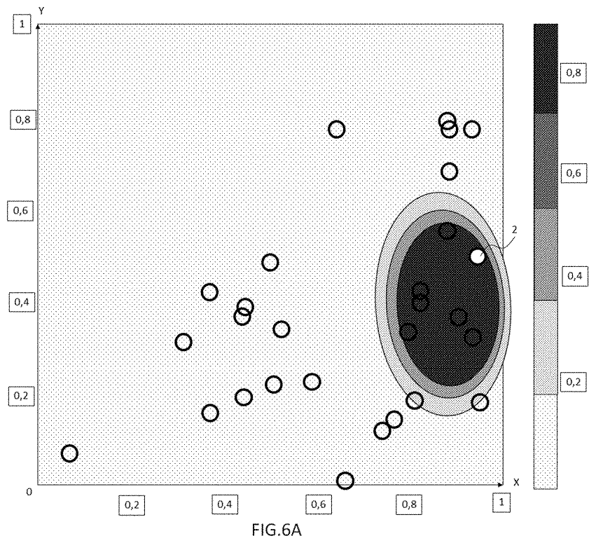

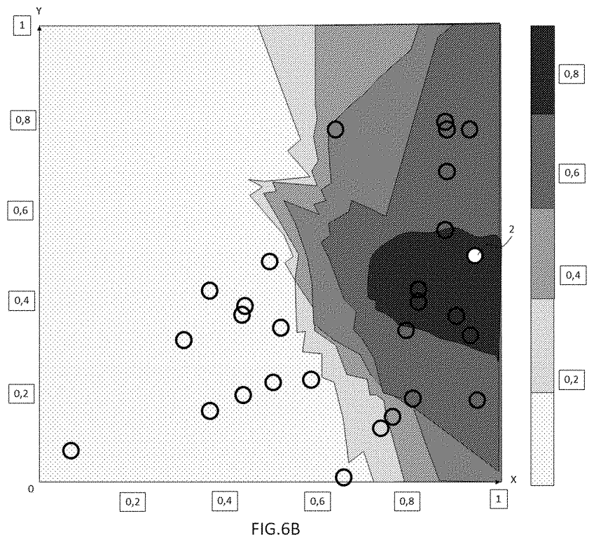

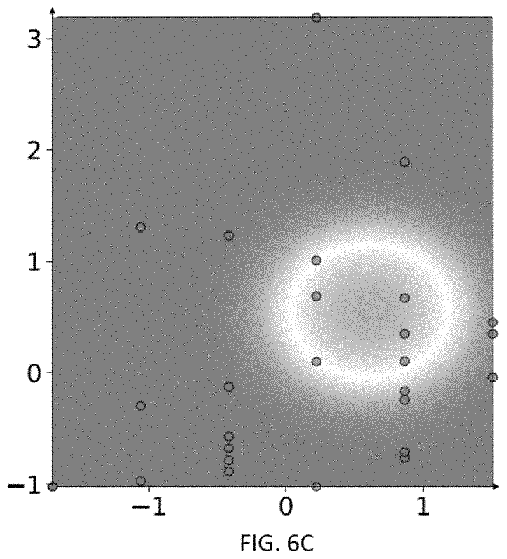

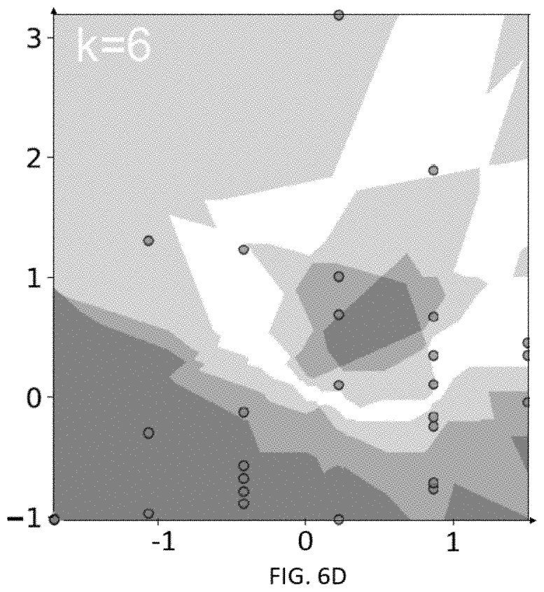

FIGS. 6A, 6B, 6C and 6D represent examples detailed below of representations of probabilities making it possible to calculate the chances that a patient H has of waking up or not from his coma.

The graph represents a probability distribution and therefore generates an indicator in a graph without specifically allowing a diagnosis to be deduced.

According to another variant, the invention does not include the generation of a two-dimensional representation but an alternative step which comprises the calculation of a distance between a new generated point 2 and a set of points ENS₁ or ENS₂ of a given class of state.

It is then possible to define a distance, such as a Euclidean distance or any other distance that can be used within the framework of the invention.

We calculate the distance d₁=d(2, ENS₁) defining the distance between point 2 of FIG. 2 of a new patient H and the set ENS₁. On the other hand, we measure the distance d₂=d(2, ENS₂) defining the distance between point 2 in FIG. 2 of a new patient H and the set ENS₂.

The two distances d₁, d₂ are then compared and a coefficient is assigned to point 2 which can be, for example, weighted by the measure of the distance or a ratio of the two distances d₁, d₂. The coefficient can also be interpreted as a probability.

According to one embodiment, the method of the invention comprises a step of defining so-called survival regions. This step improves the precision of the calculation of the probability that will be assigned to a new point generated in the graph. It also allows the distance to be weighted when the latter is used.

FIGS. 6A and 6C represent a first embodiment in which a statistical classifier is applied in order to be able to attribute a probability at any point on the map, or any point that would be added to the two-dimensional representation Ox and Oy as defined previously with P₁ and P₂. In FIG. 6A, the two-dimensional representation is based on the coordinate system {s(X), R(X, Y)} associated with responses to non-deviant stimuli, and in FIG. 6C it is based on the coordinate system {N_{E}, |ΔV|} associated with responses to deviant stimuli. According to this embodiment, a Bayesian classification based on the assumption that each class is independent, that is to say without statistical correlation, is used. In the exemplary embodiment shown, the number of classes is equal to two. These two classes correspond, for example, to the “deceased patient” or “living patient” case.

In the case of a Gaussian estimator, the average and the estimated covariance matrix over the two classes C₁ (living patient) and C₂ (deceased patient) are shown. The probability of each of the classes is calculated empirically using, for example, a maximum likelihood estimator:

$$P ( {C}_{1} ) = \frac{{n}_{s}}{{n}_{s} + {n}_{d}}$$

where nₛ is the number of patients H who survived and n_{d} is the number of patients H who died. The points X=(x,y) associated with the patients who survived follow a multidimensional normal distribution.

The probability associated with a point X=(x,y) is therefore:

$$P ( X ∈ {C}_{1} ) = \frac{{e}^{\frac{- 1}{2} {( X - {μ}_{1} )}^{T} {Σ}_{1}^{- 1} ( X - {μ}_{1} )}}{2 π {\vert {Σ}_{1} \vert}^{\frac{1}{2}}}$$

where the mean μ₁ and the variance Σ₁ are calculated on the sample of the data base from surviving patients:

$${μ}_{1} = \frac{{\sum }_{Patient {inC}_{1}} {X}_{i}}{{n}_{s}} and {Σ}_{1} = \frac{1}{{n}_{s}} {\sum }_{Patient {inC}_{1}} ( {X}_{i} - {μ}_{1} ) {( {X}_{i} - {μ}_{1} )}^{T}$$

A similar calculation is used to calculate the mean and variance of the deceased patients H database.

Using Bayes' rules, the conditional probability of having XϵC₁ and having the coordinates of the point associated with a subject (patient) equal to x, with a probability for the class C₁ (living patient), is given by:

$$π = \frac{{n}_{s}}{{n}_{s} + {n}_{d}}$$

The following conditional probability follows:

$$p ( patient ∈ {C}_{1} \vert {X}_{patient} = x ) = \frac{1}{\begin{matrix}1 + \frac{1 - π}{π} \frac{{\vert {Σ}_{1} \vert}^{\frac{1}{2}}}{{\vert {Σ}_{2} \vert}^{\frac{1}{2}}} exp \\ ( \begin{matrix}- \frac{1}{2} {( x - {μ}_{2} )}^{T} {Σ}_{2}^{- 1} ( x - {μ}_{2} ) + \\ \frac{1}{2} {( x - {μ}_{1} )}^{T} {Σ}_{1}^{- 1} ( x - {μ}_{1} )\end{matrix} )\end{matrix}}$$

FIGS. 6B and 6D represent an alternative for establishing a probability distribution on a two-dimensional graph. In FIG. 6B, the two-dimensional representation is based on the coordinate system {s(X), R(X, Y)} associated with responses to non-deviant stimuli, and in FIG. 6D it is based on the coordinate system {N_{E}, |ΔV|} associated with responses to deviant stimuli.

The k nearest neighbors method, “k-neighbor”, consists in taking into account the k nearest neighbors by calculating a ratio for the probability of belonging to a class.

According to an exemplary embodiment, for a point y, the probability of belonging to the class C₁ of “survivors” being given, the distribution of points x is calculated as the number of neighboring points belonging to the class of “survivors” on the total of K neighboring points. This probability can be calculated empirically by the following relation:

$$p ( patient ∈ {C}_{1} \vert {X}_{patient} = x ) = \frac{{k}_{r}}{K} ,$$

where kᵣ is the number of neighboring points belonging to the class of “survivors” out of a total of K neighboring points.

The result of this analysis is a probability map such as the one illustrated in FIG. 6B. Each time a new patient H is added to the database, a new mean μ₀, μ₁ and the covariance matrices Σ₀, Σ₁ are recalculated.

Point 2 in FIGS. 6A and 6B respectively represents a point corresponding to the probability p=0.62 and p=0.83.

The method therefore makes it possible to attribute a surviving probability to a given patient, for example by visualizing the position of the point corresponding to him on a two-dimensional representation as shown in FIG. 6A, 6B, 6C or 6D.

According to one embodiment, an alternative for establishing a probability distribution on a two-dimensional graph consists in the use of a supervised learning method, in particular a support vector machine (SVM). SVM make it possible to solve discrimination problems, i.e. to decide to which class a sample belongs. According to an exemplary embodiment, SVM is used to determine the separator hyperplane which best separates the classes C₁ and C₂. In this example, the kernel trick is used to overcome the lack of a linear separator present in the species problem, consisting in reconsidering the problem in a higher dimensional space. In this higher dimensional space, the points associated with the two classes are well separated. If the classes are not well separated, a penalty is associated with each misclassified point.

In one embodiment, the k nearest neighbor method is used to classify responses to deviant stimuli.

According to one embodiment, an alternative classification method is used to classify responses to non-deviant stimuli. A weighted k nearest neighbors method can be used by adding weights relative to the distance to points in the data frame. The probability of classifying the point Pt=(N_{E}, |ΔV|) in a given class among the classes C₁ and C₂ is calculated on the basis of the number of k nearest neighbors Nk(Pt) belonging to the given class which are selected, among the set of preexisting points, from the Euclidean distance between Pt and each preexisting point weighted by a weight. According to one embodiment, the weight of each preexisting point corresponds to the inverse of the distance between Pt and this point.

According to one embodiment, the probability distribution of the point Pt associated with the patient H of belonging to the class C₁ of “survivors” is calculated for each patient H for the coordinates Pt₁ in the first system of coordinates {s(X), R(X, Y)} and for the coordinates Pt₂ in the second coordinate system {N_{E}, |ΔV|} according to the following formula:

p_{dec}(PtϵC₁|Pt)=min(p(Pt₁ϵC₁|Pt₁),p(Pt₂ϵC₁|Pt₂)),

in which the probability p(Pt₁ϵC₁|Pt₁) is estimated from the k nearest neighbors method and the probability p(Pt₂ϵC₁|Pt₂) is estimated from the k weighted nearest neighbors method.

According to one embodiment, the calculation of a probability that said state indicator 2 belongs to a predefined class of states C₁, C₂ is defined as the minimum between the probabilities estimated from the k nearest neighbors method, the k weighted nearest neighbors method, the Gaussian estimator, Bayes rules and/or the support vector machine.

According to one embodiment, the method may comprise a statistical validation step. This step is carried out by considering a predetermined number of patients H, for example 20, 30 or 40. In this data validation step, the data corresponding to the new patient is then excluded.

It is then possible to compare the probability of survival of a patient H with an actual survival.

One advantage of the method is to generate a variable which can correspond to a probability of awakening. The method when implemented by a computer can then define a prediction tool to predict the survival rate of H patients in a coma.

Although various embodiments have been described and illustrated, the detailed description should not be considered as being limited thereto. Various modifications can be made to the embodiments by those skilled in the art without departing from the true spirit and scope as defined by the claims.

1-34. (canceled)

35. A method for generating a state indicator of a given patient in a coma, said method comprising:
generating at least one auditory stimulation by the generating a sequence of auditory stimuli, said sequence producing evoked potentials in the given patient;
acquiring a first electroencephalographic signal produced by said given patient from at least one electrode;
estimating at least one pair of values corresponding to a first parameter and a second parameter extracted from the first acquired signal, comprising the estimation of a first pair of values such that the calculation of the first parameter comprises an estimate of the variance of the amplitude of the first signal in a predefined time window and the calculation of the second parameter comprises a estimation of the correlation of two segments of the first signal; and
generating a state indicator for the or each pair of values of the first and second parameters, said values defining coordinates of a point in a reference base.

36. The method according to claim 35, wherein at least one stimulation comprises at least one auditory stimuli sequence comprising at least one periodic pattern of predefined frequency.

37. The method according to claim 36, wherein the calculation of the first parameter of the first pair of values comprises:
(a) filtering the first acquired signal;
(b) segmenting of the first signal according to a first time window of 1 second in order to generate a plurality of epochs synchronized with the predefined frequency, said epochs being averaged over the same time window;
(c) extracting an averaged epoch in a second predefined time window ranging from 20 ms to 320 ms, each of said epochs having a predefined duration and being synchronized with a stimulus of at least one sequence of auditory stimuli;
(d) calculation of the variance in amplitude of the averaged signal over the second predefined window.

38. The method according to claim 35, wherein the calculation of the second parameter of the first pair of values comprises:
selecting of at least a first and a second segment of signal from the acquired electroencephalographic signal, said first and second segments having the same duration;
performing a first segmentation of the first segment of signal over a third time window generating a plurality of epochs of duration corresponding to the duration of the third window, each of said epochs having a predefined duration and being synchronized with a stimulus of at least one sequence of auditory stimuli;
performing a second segmentation of the second segment of signal over the third time window generating a plurality of epochs of duration corresponding to the duration of the third window, each of said epochs having a predefined duration and being synchronized with a stimulus of at least one sequence of auditory stimuli;
generating of a first signal resulting from the averaging of the epochs of the first segmentation and generation of a second signal resulting from the averaging of the epochs of the second segmentation; and
generating of the second parameter from the calculation of the time correlation between the first average signal and the second average signal over a fourth time window ranging from 20 ms to 320 ms.

39. The method according to claim 35, wherein the estimation step comprises estimating a second pair of values corresponding to a first parameter and a second parameter extracted from the first acquired signal, such that the calculation of the first parameter comprises an estimate of the number of local extremums in the first signal in a predefined time window and calculating the second parameter comprises the sum of the absolute values of the differences in potential value of the first signal between two successive local extremes in a predefined time window, allowing the generation of a second state indicator defined by the second pair of values of the first and second parameters.

40. The method according to claim 38, wherein the first time window and the third time window have the same duration, corresponding to the inverse of the predefined frequency.

41. The method according to claim 35, wherein generating the state indicator for said given patient comprises calculating a probability that said state indicator belongs to a predefined class of states from a Gaussian estimator, Bayes rules and/or a support vector machine.

42. The method according to claim 35, wherein generating the state indicator for said given patient comprises calculating a probability that said state indicator belongs to a predefined state class using the k nearest neighbors method.

43. The method according to claim 35, wherein generating the state indicator for said given patient comprises calculating a probability that said state indicator belongs to a predefined class of states from the minimum between the probabilities estimated from the k nearest neighbors method, the weighted k nearest neighbors method, the Gaussian estimator, the rules of Bayes and/or the support vector machine.

44. The method according to claim 35, wherein generating at least one state indicator for said given patient comprises calculating a probability that the first state indicator belongs to a predefined state class from the k nearest neighbors method and the calculation of a probability that the second state indicator belongs to a predefined state class using the weighted k nearest neighbors method.

45. The method according to claim 41, wherein the probability that the patient belongs to a predefined state class is estimated as the minimum between the probability calculated for the first indicator from of the k nearest neighbors method and the probability calculated for the second state indicator from the weighted k nearest neighbors method.

46. The method according to claim 35, further comprises measuring a first distance between said state indicator and a first set of points having coordinates represented in the same reference base and for measuring a second distance between said state indicator and a second set of points having coordinates represented in the same baseline.

47. The method according to claim 35, further comprises comparing the first distance and the second distance.

48. The method according to claim 35, wherein the state indicator is associated with a probability calculated from a probabilities classification model or a supervised learning classification method.

49. The method according to claim 48, wherein the classification comprises two classes.

50. The method according to claim 35, wherein the steps are repeated for a second patient and/or any other patient associated with an electroencephalographic signal or with recorded data from a database and further include:
(a) generating a graph for a plurality of coordinates from a patients population, in which each patient is associated with coordinates [(P₁); (P₂)]ᵢ;
(b) identifying at least one region of interest of the graph for which a subset of selected patients shares the same class of the predefined classification;
(c) associating a probability with one of the classification classes for each of the identified regions of interest;
(d) generating a probability associated with the state indicator of the first patient on the basis of the coordinates [(P₁); (P₂)].

51. A method for constituting a baseline for evaluating a probability of awakening of a patient in a coma, said method comprising:
(a) generating a state indicator of a new patient according to the file method according to claim 35;
(b) recording the set of coordinates in a memory, said coordinates being associated with a state indicator of said first patient;
(c) updating a set of wake-up reference data stored in a memory.

52. A device for generating a state indicator of a given patient in a coma, comprising:
a stimulation module configured to generate at least one auditory stimulation by the generation of a sequence of auditory stimuli, said sequence producing evoked potentials in the given patient;
an acquisition module configured for the acquisition of a first electroencephalographic signal produced by said given patient from at least one electrode;
a calculation module configured for the estimation of at least one pair of values corresponding to a first parameter and a second parameter extracted from the first acquired signal, comprising the estimation of a first pair of values such that the calculation of the first parameter comprises an estimate of the amplitude variance of the first signal in a predefined time window and the calculation of the second parameter comprises an estimate of the correlation of two segments of the first signa; and
a generation module configured to generate a state indicator for the or each pair of values of the first and second parameters, said values defining coordinates of a point in a base of reference.

53. The device according to claim 52, wherein the generation module is configured to generate the state indicator for said given patient from the calculation of a probability that the first state indicator belongs to a predefined state class from the k nearest neighbors method and the calculation of a probability that the second state indicator belongs to a predefined state class from the weighted k nearest neighbors method.

54. The device according to claim 52, wherein a calculation module is configured to repeat the steps for a second patient and/or any other patient associated with an electroencephalographic signal or with recorded data of a database and to additionally implement the steps of:
(a) generating a graph for a plurality of coordinates from a patients population, in which each patient is associated with coordinates [(P₁);(P₂)]ᵢ;
(b) identification of at least one region of interest of the graph for which a subset of selected patients shares the same class of the predefined classification;
(c) association of a probability with one of the classification classes for each of the identified regions of interest;
(d) generation of a probability associated with the state indicator of the first patient on the basis of the coordinates [(P₁);(P₂)].

55. A system comprising a generator of auditory stimuli emitted with a predefined period for a predefined duration and a set of electrodes for measuring a cerebral electrical activity of a patient, the system comprising a device according to claim 52 for generating a state indicator of said patient.
